(19) United States
(12) Patent Application Publication
(10) Pub. No.: US 2023/0181545 A1
(43) Pub. Date: Jun. 15, 2023

(54) CONTROLLED-RELEASE PHARMACEUTICAL FORMULATIONS FOR TREATMENT OF INTESTINAL INFECTIONS

(71) Applicant: DPL PHARMA S.P.A., Rozzano (MI) (IT)

(72) Inventors: Massimo Pedrani, Melide (CH); Chiara Conti, Vigolo Marchese (PC) (IT); Salvatore Agostino Giammillari, Rozzano (MI) (IT); Giuseppe Maccari, Voghera (PV) (IT)

(73) Assignee: DPL PHARMA S.P.A., Rozzano (MI) (IT)

(21) Appl. No.: 17/906,523

(22) PCT Filed: Mar. 17, 2021
(86) PCT No.: PCT/IB2021/052206

§ 371 Date: Sep. 16, 2022

(30) Foreign Application Priority Data
Mar. 18, 2020 (IT) 102020000005782

(51) Int. Cl. A61K 31/4375; A61K 47/38; A61K 9/20; A61K 9/16

(52) U.S. Cl. CPC A61K 31/4375; A61K 9/1664; A61K 9/2027; A61K 47/38

(57) ABSTRACT

Disclosed are solid oral controlled-release pharmaceutical compositions of antibiotics/ antibacterials comprising a core consisting of a complex monolithic matrix comprising at least one low/medium viscosity hydroxypropyl methylcellulose, at least one medium/high viscosity hydroxypropyl methylcellulose, one or more methacrylic polymers or copolymers and/or cellulose acetate phthalate and/or hydroxypropyl methylcellulose acetate succinate or shellac, and an outer coating of said core consisting of a layer comprising ethylcellulose, or of a gastroresistant layer or of a layer comprising ethylcellulose coated in turn with gastroresistant polymers. The compositions of the invention enable the frequency of administration to be reduced, and the release of the medicament in particular sites of the gastrointestinal tract to be controlled.

The invention relates to solid oral controlled-release pharmaceutical formulations of antibacterials/antibiotics for the treatment of intestinal infections.

### BACKGROUND OF THE INVENTION

Various disorders of the gastrointestinal system require treatment with antibiotics/antibacterials able to concentrate in the intestinal lumen wherein their antibacterial activity takes place. Examples of said medicaments include aminoglycoside antibiotics, rifaximin, rifamycin SV and the salts thereof, rifabutin, vancomycin, teicoplanin, bacitracin, metronidazole and ciprofloxacin. In particular rifaximin, a semisynthetic derivative of rifamycin, is widely used in clinical practice due to its efficacy and high level of tolerability. Said antibiotics/antibacterials are indicated for the treatment of various conditions characterised by bacterial infections, possibly associated with inflammatory symptoms, with consequent alteration of the intestinal bacterial flora (microbiota). Examples of said conditions include Crohn’s disease, irritable bowel syndrome, ulcerative colitis, travellers’ diarrhoea, diverticulitis, hepatic encephalopathy, dysbiosis, and small intestinal bacterial overgrowth syndrome (SIBO).

Other disorders of the gastrointestinal tract, associated with the treatment of Helicobacter pylori, can be treated with formulations of antibiotics combined with antacids, proton pump inhibitors, and P-CAB (Potassium Competitive Acid Blockers). Antibiotics such as ciprofloxacin, amoxicillin and rifabutin can be conveniently formulated in modified- and controlled-release therapeutic systems to improve their efficacy and patient compliance.

The design of oral administration forms of said antibacterials/antibiotics must take account of various issues associated with pH variations in the gastrointestinal tract, and with transit and gastric voiding times, which are highly variable, depending on the patient’s condition (high individual variability) and the type and amount of food eaten before administration. Transit times depend partly on the pharmaceutical form, and can range from a few minutes to 24-48 hours.

Rifamycin SV and rifaximin SV sodium salt are currently marketed and/or under development for the treatment of travellers’ diarrhoea (TD), HE and infectious IBS-D; rifaximin is formulated in immediate-release pharmaceutical form, while rifamycin SV sodium salt is formulated in a gastroresistant delayed form to prevent its exposure to the gastric environment, the acid pH of which could compromise its stability. The currently available formulations are characterised by a high unit dose and high frequency of administration (2-4 times a day).

Controlled-release formulations of rifamycin SV useful for the treatment of infections of the large intestine, in particular the colon, are described in US 8,263,120 and WO20139883.

Chintan Parmar et al. in J. Drug Delivery Sci. and Technol., Vol 44, 01.04.2018, 388-398 describe two-layer tablets with an enteric coating for colon-specific administration of mesalazine.

### DESCRIPTION OF THE INVENTION

It has now been found that the activity of oral antibiotics can be effectively modulated, reducing their frequency of administration and controlling their release in particular sites of the gastrointestinal tract, by using complex matrices consisting of a combination of polymers with different characteristics.

In particular, it has been found that combining at least two types of hydroxypropyl methylcellulose having different viscosities with methacrylic polymers or copolymers and/or cellulose resins or esters or shellac makes it possible to prepare formulations that overcome the limitations of the previously known formulations, especially as regards the variability of dissolution profiles over time, with low relative standard deviation (RSD) values.

The use of complex monolithic matrices also gives rise to pharmaceutical forms of intestinal antibacterials which can be administered once a day, with suitably modulated release in the gastrointestinal tract, for the treatment of various intestinal disorders.

The solid oral controlled-release pharmaceutical compositions according to the invention comprise a core consisting of a complex monolithic matrix comprising at least one low/medium viscosity hydroxypropyl methylcellulose, at least one medium/high viscosity hydroxypropyl methylcellulose, one or more methacrylic polymers or copolymers and/or cellulose acetate phthalate and/or hydroxypropyl methylcellulose acetate succinate or shellac, and an outer coating of said core consisting of a layer comprising ethylcellulose, or of a gastroresistant layer or of a layer comprising ethylcellulose coated in turn with gastroresistant polymers.

### DETAILED DESCRIPTION OF THE INVENTION

The solid oral controlled-release pharmaceutical compositions according to the invention comprise a core containing an antibacterial for intestinal infections and an outer coating of said core, wherein:
a) the core comprises:
   (i) a monolithic matrix containing the antibacterial, at least one hydroxypropyl methylcellulose having a viscosity ranging between 3 and 5000 mPa.s 2% in H₂O at 20° C., at least one hydroxypropyl methylcellulose having a viscosity ranging between 13500 and 280000 mPa.s 2% in H₂O at 20° C., at least one or more methacrylic polymers/copolymers and/or shellac, cellulose acetate phthalate, hydroxypropyl methylcellulose acetate succinate, or
   (ii) a monolithic matrix as defined above adjacent to an immediate-release layer comprising the same or other antibacterial as contained in the monolithic matrix;
b) the coating consists of a layer comprising ethylcellulose or of a gastroresistant layer or of a layer comprising ethylcellulose which in turn is coated with gastroresistant polymers.

The core can consist of a complex monolithic matrix (i) or a bi-layer system consisting of a complex monolithic matrix (i) adjacent to an immediate-release layer comprising the same antibacterial as contained in the monolithic matrix or a different antibacterial.

The coating consists of a layer comprising ethylcellulose or, in another embodiment of the invention, coating b) consists of a layer comprising ethylcellulose coated with gastroresistant polymers.

In yet another embodiment of the invention, the coating consists of a gastroresistant layer.

The acrylic/methacrylic polymers or copolymers of matrix (i) are preferably selected from a mixture of pH-independent methacrylic ester copolymers, pH-independent ammonium alkyl methacrylate copolymers; amino alkyl methacrylate copolymers soluble up to pH 5.0, methacrylic acid copolymers soluble at pH ≥ 5.5, methacrylic acid copolymers soluble at pH 6.0-7.0; and pH-dependent methacrylic acid copolymers soluble at pH ≥ 7.0.

According to one embodiment of the invention, two or more polymers or copolymers can be combined, or acrylic polymers or copolymers are combined with shellac, or the latter can replace said acrylic polymers/copolymers.

The gastroresistant coating can be the conventional type, and typically comprises methacrylic acid copolymers soluble at pH ≥ 5.5. Examples of said copolymers are available on the market (Eudragit). Combinations of polymethacrylate L100 with polymethacrylate S100 at the ratio of 1:10-10:1 (preferably 1:1), polymethacrylates L 100/55 soluble at pH ≥ 5.5, shellac or cellulose acetate phthalates/acetate succinates are preferred.

In the compositions of the invention, hydroxypropyl methylcellulose having a viscosity ranging between 3 and 5000 mPa.s 2% in H₂O at 20° C. constitutes 1 to 20% of the weight of the core, hydroxypropyl methylcellulose having a viscosity ranging between 13500 and 280000 mPa.s 2% in H₂O at 20° C. constitutes 1 to 20% of the weight of the core, and methacrylic polymer/copolymer constitutes 0.1 to 20% of the weight of the core (preferably 0.1% to 2%).

Hydroxypropyl methylcellulose having a viscosity ranging between 3.0 and 5000 mPa.s 2% in H₂O at 20° C. is available on the market under the names of Methocel K3LV, K100 LV, E5 premium and K4M.

Hydroxypropyl methylcellulose having a viscosity ranging between 13500 and 280000 mPa.s 2% in H₂O at 20° C. is available on the market under the names of Methocel K15 M, K100 M and K200 M. Methocel K15 M and K100 M are preferred.

Ethylcellulose is present in the core-coating layer in percentages ranging from 1% to 20% of the weight of the core; preferably 5%.

The matrix core can comprise conventional excipients such as diluents (microcrystalline cellulose, starches, sugars, phosphate salts - hydrated and anhydrous mono/dibasic sodium phosphate), binders (PVP, starches, cellulose, dextrins, maltodextrins, low-viscosity cellulose), glidants (colloidal silicon dioxides, talc), lubricants (Mg stearate, fumaryl stearate, stearic acid, glyceryl behenate), disintegrating agents (croscarmellose, sodium starch glycolate, crosslinked polyvinylpyrrolidone, starches) and other functional excipients (waxes, polycarbophil, carbomer, glycerides).

The matrix is prepared by processes of partition and direct compression, dry granulation, compacting, wet granulation, melting and extrusion.

Powders, granules, microgranules, pellets, mini-tablets, tablets, capsules, sachets and sticks can thus be obtained.

The resulting matrix/mini-matrix can then be coated with a gastroresistant film containing pH-dependent polymers that prevent release for at least 2 hours under pH conditions < 1.2-5.5. The following can be used for this purpose: pH-dependent methacrylic acid copolymers soluble at pH ≥5.5 (L 100-55/L 30 D-55); pH-dependent methacrylic acid copolymers soluble at pH 6.0-7.0 (L 100/L 12.5); pH-dependent methacrylic acid copolymers soluble at pH ≥ 7.0 (S 100/S 12.5/FS 30D); shellac; cellulose acetate phthalate; hydroxypropyl methylcellulose acetate succinate.

At a third stage, a core coating can be applied which is alternative and/or additional to and beneath the gastroresistant coating with pH-independent polymers (ethylcellulose or hydroxypropyl methylcellulose with different viscosities), which act as membranes delaying the passage of the ingredient loaded into the matrix/mini-matrix core following contact with biological fluids.

The matrix is coated with an amount of polymer sufficient to guarantee that it remains intact in gastric and enteric juices for at least 2-4 hours before the release of the active ingredient from the core (lag time). To reduce the impact of variable gastric voiding times, a further (pH-dependent) gastroresistant coating can be applied outside the (pH-independent) matrix core and outside the (pH-independent) cellulose film coating, to further delay contact between the biological fluids and the modified-release core (extended release).

In this way the system prevents early release during the stomach-jejunum transit time, initiating the modulated-release programme lasting up to 24 hours and ensuring homogeneous distribution of the active ingredient in the duodenum, ileum and distal ileum and in the ascending, transverse and descending tracts of the large intestine.

The use of hydrophilic polymers with different rheological characteristics (viscosity/swelling properties) combined with pH-dependent and/or pH-independent polymers allows the release to be modulated for between 8 and 24 hours. If desired, a modified-, controlled-release core can be combined with an immediate-release layer (bi-layer and/or tri-layer matrix/mini-matrix); a system thus designed gives results of “therapeutic equivalence” or different levels of therapeutic efficacy.

Examples of active ingredients which can be advantageously formulated according to the invention include rifaximin, rifampicin, rifamycin sv, oral beta-lactams and macrolides, and quinolines such as ciprofloxacin and metronidazole. The formulations of the invention are particularly suitable to optimise the pharmacological effect of medicaments which have an unfavourable profile in terms of compliance, because of the large number of daily administrations, and the side effects.

The formulations of the invention can have different release profiles:
release from distal ileum (delay from pH ≥6.8-7.2); then gradual, constant release for several hours;
release from duodenum (delay from pH ≥5); then a further release lag time of 2-3 hours, and finally gradual, constant release for several hours;
release from duodenum (delay from pH ≥5); then gradual, constant release for several hours;
gradual, constant release from stomach (extended and controlled release);
pH-independent delayed release of 2-3 hours followed by gradual, constant release for several hours;
immediate release of a portion of medicament, and controlled, extended release for several hours of a second portion of medicament.

The release of the active ingredient from the compositions of the invention can be modulated according to the disorder/symptoms to be treated.

For example, for the treatment of dysbiosis, Crohn’s disease, non-alcoholic steatohepatitis and hepatic encephalopathy, the antibacterial is opportunely released from the proximal ileum to the colon by means of matrices soluble at ≥pH 5.5.

For small intestinal bacterial overgrowth syndrome, in addition to release from the proximal ileum, it is appropriate for a portion of the medicament to be immediate-release.

For treatment of travellers’ diarrhoea, irritable bowel syndrome and ulcerating colitis, formulations able to release the medicament from the terminal ileum via matrices soluble at ≥pH 5.5, and then in the colon, after a lag time, from matrices soluble at ≥pH 7.2, can conveniently be used.

For the treatment of diverticular disease or diverticulitis, formulations able to release the medicament into the colon from matrices soluble at ≥pH 7.2 and ≥pH 5.5 after a 3 h lag time can conveniently be used.

The invention is described in detail in the examples below.

### Example 1

7.5 Kg of rifaximin, 650 g of hydroxypropyl methylcellulose (HPMC 100 Iv), 330 g of hydroxypropyl methylcellulose (HPMC K1 00 M), 500 g of microcrystalline cellulose, 50 g of talc, 70 g of glyceryl palmitostearate, 50 g of silicon dioxide, 25 g of polymethacrylate L100 and 25 g of polymethacrylate S 100 are standardised through a 12 mesh screen. After normalisation, 7.5 Kg of rifaximin, 325 g of hydroxypropyl methylcellulose (HPMC 1001v), 165 g of hydroxypropyl methylcellulose (HPMC K100M), 250 g of microcrystalline cellulose, 25 g of talc, 35 g of glyceryl palmitostearate, 25 g of silicon dioxide, 12.5 g of polymethacrylate L100 and 12.5 g of polymethacrylate S 100 are taken up, placed in a mixer and mixed for 15 minutes; then loaded into a compacter; suitably compacted and standardised through a 12 mesh screen. An external phase comprising 325 g of hydroxypropyl methylcellulose (HPMC 1001v), 165 g of hydroxypropyl methylcellulose (HPMC K100M), 250 g of microcrystalline cellulose, 25 g of talc, 35 g of glyceryl palmitostearate, 25 g of silicon dioxide, 12.5 g of polymethacrylate L100 and 12.5 g of polymethacrylate S 100 is added to said mixture, and the two phases are mixed. The mixture is then compressed in a tablet press to obtain tablets weighing 92 mg each.

The resulting tablets are film-coated with a gastroresistant solution/suspension based on 480 g of polymethacrylate L100, 480 g of polymethacrylate S100, 150 g of talc, 20 g of titanium dioxide and 70 g of triethyl citrate, to obtain a tablet with a mean weight of 104 mg.

At disintegration and dissolution tests at pH 1, the tablets remain intact for at least 2 hours, with release below 1%; at the dissolution test at pH ≥ 6.4 they showed the following release profile: not more than 1% after 1 hour, at pH 7.2 not more than 30% after 1 hour, and not more than 35% after 2 hours; the value must be > 80% after 6 hours; and 100% after 10 hours.

### Example 2

The process continues similarly to Example 1, replacing HPMC 100 lv with HPMC K4M, then adding polymethacrylate L100/55 into the matrix, instead of polymethacrylate L100 and polymethacrylate S100. The coating is performed with ethylcellulose and polymethacrylate L100/55 vs. polymethacrylate L100 and polymethacrylate S100.

At disintegration and dissolution tests at pH 1, the tablets remain intact for at least 2 hours, with release below 1%; at the dissolution test at pH ≥ 5.5 they showed the following release profile: not more than 1% after 1 hour, at pH 7.2 not more than 5% after 1 hour, and not more than 10% after 2 hours; not more than 30% after 4 hours; not more than 50% after 6 hours; the value must be ≥80% after 8 hours; and 100% after 12 hours.

### Example 3

The process is conducted similarly to Example 1, replacing HPMC K100M with HPMC K15M, then adding polymethacrylates RL100 and RS100 into the matrix instead of polymethacrylates L100 and S100. The coating is performed with HPMC 5 Premium and polymethacrylate L100/55 vs. polymethacrylate L100 and polymethacrylate S100.

At disintegration and dissolution tests at pH 1, the tablets remain intact for at least 2 hours, with release below 1%; at the dissolution test at pH ≥ 5.5 they showed the following release profile: not more than 1% after 1 hour, at pH 7.2 not more than 30% after 1 hour, and not more than 50% after 2 hours; not more than 70% after 4 hours; not more than 80% after 6 hours; the value must be ≥ 80% after 8 hours; and 100% after 12 hours.

### Example 4

The process is conducted similarly to Example 3, replacing the entire amount of polymethacrylate L100/55 with HPMC E 5 Premium in the film coating.

At dissolution tests at pH 1, the tablets showed the following release profile: release lower than 20% after 2 hours; at the dissolution test at pH ≥ 6.4 they showed the following release profile: not more than 40% after 1 hour, at pH 7.2 not more than 60% after 1 hour, and not more than 70% after 2 hours; the value must be > 80% after 6 hours; and 100% after 12 hours.

### Example 5

The process is conducted similarly to Example 2, reducing the total amount of raw materials to obtain mini-tablets with a diameter of 3 mm vs 5 mm and replacing polymethacrylate L100/55 in the matrix with polymethacrylates RL100 and RS100. The coating is performed with ethylcellulose alone, eliminating polymethacrylate L100/55.

At disintegration and dissolution tests at pH 1, the tablets remain intact for at least 2 hours, with release below 5%; at the dissolution test at pH ≥ 6.4 they showed the following release profile: not more than 10% after 1 hour, at pH 7.2 not more than 30% after 1 hour, and not more than 50% after 2 hours; not more than 70% after 4 hours; not more than 80% after 6 hours; the value must be > 80% after 8 hours; and 100% after 12 hours.

### Example 6

The process is conducted similarly to Example 5, producing mini-tablets with a diameter of 3 mm by replacing polymethacrylates RL100 and RS100 in the matrix with polymethacrylate L100/55. The coating is performed with polymethacrylate L100/55, eliminating ethylcellulose.

At disintegration and dissolution tests at pH 1, the tablets remain intact for at least 2 hours, with release below 1%; at the dissolution test at pH ≥ 5.5, they exhibit the following release profile: not more than 20% after 1 hour; at pH 7.2 not more than 30% after 1 hour, not more than 50% after 2 hours; not more than 70% after 4 hours; not more than 80% after 6 hours; the value must be > 80% after 8 hours; and 100% after 12 hours.

### Example 7

The process is conducted similarly to Example 6, producing mini-tablets with a diameter of 3 mm by replacing polymethacrylate L100/55 in the matrix with polymethacrylates L100 and S100. The coating is performed with polymethacrylates L100 and S100, eliminating polymethacrylate L100/55.

At disintegration and dissolution tests at pH 1, the tablets remain intact for at least 2 hours, with release below 1%; at the dissolution test at pH ≥ 6.4 they exhibit the following release profile: not more than 1% after 1 hour, at pH 7.2 not more than 20% after 1 hour, and not more than 30% after 2 hours; the value must be > 80% after 6 hours; and 100% after 12 hours.

### Example 8

The process is conducted similarly to Example 7, producing mini-tablets with a diameter of 3 mm by replacing polymethacrylates L100 and S100 in the matrix with polymethacrylates RL100 and RS100. The coating is performed with HPMC E5 Premium instead of polymethacrylates L100 and S100.

At dissolution tests at pH 1, the tablets showed the following release profile: release lower than 20% after 2 hours; at the dissolution test at pH ≥ 6.4 they showed the following release profile: not more than 40% after 1 hour, at pH 7.2 not more than 60% after 1 hour, and not more than 70% after 2 hours; the value must be > 80% after 8 hours; and 100% after 12 hours.

### Example 9

The process is conducted similarly to Example 8, increasing the amount of the raw materials and producing mini-tablets with a diameter of 5 mm, and adding shellac into the matrix. The coating is performed by adding shellac to HPMC E5 Premium.

At dissolution tests at pH 1, the tablets showed the following release profile: release lower than 1% after 2 hours; at the dissolution test at pH ≥ 6.4 they showed the following release profile: not more than 20% after 1 hour, at pH 7.2 not more than 60% after 1 hour, and not more than 70% after 2 hours; the value must be > 80% after 8 hours; and 100% after 12 hours.

### Example 10

7.5 Kg of rifaximin, 350 g of hydroxypropyl methylcellulose (HPMC K4M), 175 g of hydroxypropyl methylcellulose (HPMC K15M), 1.075 Kg of microcrystalline cellulose, 90 g of talc, 60 g of glyceryl palmitostearate, 60 g of silicon dioxide, 10 g of polymethacrylate RL100, 10 g of polymethacrylate RS 100, 335 g of crospovidone and 335 g of croscarmellose are standardised through a 12 mesh screen.

After normalisation, 3.75 Kg of rifaximin, 175 g of hydroxypropyl methylcellulose (HPMC K15M), 750 g of microcrystalline cellulose, 7.5 g of talc, 15 g of glyceryl palmitostearate, 5 g of silicon dioxide, 10 g of polymethacrylate RL100 and 10 g of polymethacrylate RS 100 are taken up, placed in a mixer and mixed for 15 minutes; then loaded into a compacter; suitably compacted and standardised through a 12 mesh screen.

7.5 g of talc, 15 g of glyceryl palmitostearate and 5 g of silicon dioxide are added to this mixture. The mixture is then homogenised for at least 15 minutes. This mixture will form part of the first, controlled-release layer of the mini-tablet, consisting of a single 51 mg layer.

3.75 Kg of rifaximin, 325 g of microcrystalline cellulose, 335 g of crospovidone, 335 g of croscarmellose, 37.5 g of talc, 15 g of glyceryl palmitostearate and 25 g of silicon dioxide are taken up separately, placed in a mixer and mixed for 15 minutes; then loaded into a compacter; suitably compacted and standardised through a 12 mesh screen.

7.5 g of talc, 15 g of glyceryl palmitostearate and 25 g of silicon dioxide are added to this mixture. The mixture is then homogenised for at least 15 minutes. This mixture will form part of the second, immediate-release layer of the mini-tablet, which comprises a single 49 mg layer.

The two separate mixtures are then compressed to obtain a 5 mm double-layer mini-tablet weighing 100 mg. The resulting mini-tablets are then film-coated with a solution/suspension containing 540 g of HPMC E5 Premium, 200 g of talc, 60 g of titanium dioxide and 200 g of triethyl citrate, to obtain a mini-tablet with a mean weight of 110 mg.

The mini-tablets subjected to dissolution tests at pH 1 after 1 hour exhibit release below 50%; at pH ≥ 6.4 they showed release ≤ 60% after 1 hour; at pH 7.2 release ≤ 70% after 1 hour; release ≤ 80% after 6 hours, not more than 85% after 8 hours; the value must be 100% after 12 hours.

### Example 11

7.5 Kg of rifamycin sv sodium salt, 650 g of hydroxypropyl methylcellulose (HPMC K4M), 330 g of hydroxypropyl methylcellulose (HPMC K15M), 500 g of microcrystalline cellulose, 60 g of talc, 70 g of glyceryl palmitostearate, 50 g of silicon dioxide, 30 g of polymethacrylate L100, 30 g of polymethacrylate S 100 and 380 g of ascorbic acid are standardised through a 12 mesh screen. After normalisation, 7.5 Kg of rifamycin sv sodium salt, 325 g of hydroxypropyl methylcellulose (HPMC K4M), 165 g of hydroxypropyl methylcellulose (HPMC K15), 250 g of microcrystalline cellulose, 30 g of talc, 35 g of glyceryl palmitostearate, 25 g of silicon dioxide, 15 g of polymethacrylate L100, 15 g of polymethacrylate S 100 and 380 g of ascorbic acid are taken up, placed in a mixer and mixed for 15 minutes; then loaded into a compacter; suitably compacted and standardised through a 12 mesh screen. An external phase comprising 325 g of hydroxypropyl methylcellulose (HPMC K4M), 165 g of hydroxypropyl methylcellulose (HPMC K15), 250 g of microcrystalline cellulose, 30 g of talc, 35 g of glyceryl palmitostearate, 25 g of silicon dioxide, 15 g of polymethacrylate L100 and 15 g of polymethacrylate S 100 is added to said mixture, and the two phases are mixed. The mixture is then compressed in a tablet press to obtain tablets weighing 96 mg each.

The resulting tablets are film-coated with a gastroresistant solution/suspension based on 480 g of polymethacrylate L100, 480 g of polymethacrylate S100, 150 g of talc, 120 g of titanium dioxide and 70 g of triethyl citrate, to obtain a tablet with a mean weight of 109 mg.

At disintegration and dissolution tests at pH 1, the tablets remain intact for at least 2 hours, with release below 1%; at the dissolution test at pH ≥ 6.4 they showed the following release profile: not more than 1% after 1 hour, at pH 7.2 not more than 30% after 1 hour, and not more than 35% after 2 hours; the value must be > 80% after 6 hours; and 100% after 10 hours.

### Example 12

7.5 Kg of rifamycin sv sodium salt, 650 g of hydroxypropyl methylcellulose (HPMC 100 Iv), 330 g of hydroxypropyl methylcellulose (HPMC K15M), 500 g of microcrystalline cellulose, 50 g of talc, 70 g of glyceryl palmitostearate, 50 g of silicon dioxide, 50 g of polymethacrylate L100-55 and 380 g of ascorbic acid are standardised through a 12 mesh screen. After normalisation, 7.5 Kg of rifamycin sv sodium salt, 325 g of hydroxypropyl methylcellulose (HPMC 100 Iv), 165 g of hydroxypropyl methylcellulose (HPMC K15M), 250 g of microcrystalline cellulose, 25 g of talc, 35 g of glyceryl palmitostearate, 25 g of silicon dioxide, 25 g of polymethacrylate L100-55 and 380 g of ascorbic acid are taken up, placed in a mixer and mixed for 15 minutes; then loaded into a compacter; suitably compacted and standardised through a 12 mesh screen. An external phase comprising 325 g of hydroxypropyl methylcellulose (HPMC 100 Iv), 165 g of hydroxypropyl methylcellulose (HPMC K15M), 250 g of microcrystalline cellulose, 25 g of talc, 35 g of glyceryl palmitostearate, 25 g of silicon dioxide and 250 g of polymethacrylate L100-55 is added to said mixture, and the two phases are mixed. The mixture is then compressed in a tablet press to obtain mini-tablets weighing 96 mg each.

The resulting tablets are first coated with a suspension/solution containing 560 g of ethylcellulose, 75 g of talc, 60 g of titanium dioxide and 38 g of triethyl citrate.

The tablets are further coated with a gastroresistant solution/suspension based on 900 g of polymethacrylate L100-55, 75 g of talc, 60 g of titanium dioxide and 35 g of triethyl citrate, to obtain a tablet with a mean weight of 115 mg.

At disintegration and dissolution tests at pH 1, the tablets remain intact for at least 2 hours, with release below 1%; at the dissolution test at pH ≥ 5.5, they showed the following release profile: not more than 1% after 1 hour, at pH 7.2 not more than 5% after 1 hour, and not more than 10% after 2 hours; not more than 30% after 4 hours; not more than 50% after 6 hours; the value must be > 80% after 8 hours; and 100% after 12 hours.

### Example 13

The process is conducted similarly to Example 12, using HPMC K4M instead of HPMC 1001v and HPMC K100M instead of HPMC K15M, and adding polymethacrylates RL100 and RS100 in the matrix instead of polymethacrylate L100/55. The coating is performed by adding HPMC E5 Premium to polymethacrylate L100/55.

At disintegration and dissolution tests at pH 1, the tablets remain intact for at least 2 hours, with release below 1%; at the dissolution test at pH ≥ 5.5, they showed the following release profile: not more than 10% after 1 hour; at pH 7.2 not more than 30% after 1 hour, not more than 50% after 2 hours; not more than 70% after 4 hours; not more than 80% after 6 hours; the value must be > 80% after 8 hours; and 100% after 12 hours.

### Example 14

The process is conducted similarly to Example 13, using HPMC 100 lv instead of HPMC K4M. The coating is performed with HPMC E5 Premium instead of polymethacrylate L100/55.

At the dissolution test at pH 1, the tablets showed the following release profile: release lower than 25% after 2 hours; at the dissolution test at pH ≥ 6.4 they showed the following release profile: not more than 40% after 1 hour, at pH 7.2 not more than 65% after 1 hour, and not more than 70% after 2 hours; the value must be > 80% after 6 hours; and 100% after 12 hours.

### Example 15

The process is conducted similarly to Example 14, reducing the total amount of raw materials to obtain mini-tablets with a diameter of 3 mm vs 5 mm. The coating is performed with ethylcellulose only, eliminating HPMC E5 Premium.

At disintegration and dissolution tests at pH 1, the tablets remain intact for at least 2 hours, with release below 5%; at the dissolution test at pH ≥ 6.4, they showed the following release profile: not more than 10% after 1 hour, at pH 7.2 not more than 30% after 1 hour, and not more than 50% after 2 hours; not more than 70% after 4 hours; not more than 80% after 6 hours; the value must be > 80% after 8 hours; and 100% after 12 hours.

### Example 16

The process is conducted similarly to Example 15, replacing HPMC 1001v with HPMC K4M and maintaining HPMC K100M. Polymethacrylate L100/55 is introduced into the matrix instead of polymethacrylates L100 and S100. The coating is performed with HPMC L100/55 instead of ethylcellulose.

At disintegration and dissolution tests at pH 1, the tablets remain intact for at least 2 hours, with release below 1%; at the dissolution test at pH ≥ 5.5, they showed the following release profile: not more than 15% after 1 hour; at pH 7.2 not more than 35% after 1 hour, not more than 55% after 2 hours; not more than 70% after 4 hours; not more than 80% after 6 hours; the value must be > 80% after 8 hours; and 100% after 12 hours.

### Example 17

The process is conducted similarly to Example 16, replacing HPMC K100M with HPMC K15M. Polymethacrylates L100 and S100 are introduced into the matrix instead of L100/55. The coating is performed with polymethacrylates L100 and S100 instead of HPMC L100/55.

At disintegration and dissolution tests at pH 1, the tablets remain intact for at least 2 hours, with release below 1%; at the dissolution test at pH ≥ 6.4 they showed the following release profile: not more than 1% after 1 hour, at pH 7.2 not more than 20% after 1 hour, and not more than 40% after 2 hours; the value must be > 80% after 6 hours; and 100% after 12 hours.

### Example 18

The process is conducted similarly to Example 17. Polymethacrylates RL100 and RS100 are introduced into the matrix instead of polymethacrylates L100 and S100. The coating is performed with HPMC E 5 Premium instead of polymethacrylates L100 and S100.

At the dissolution test at pH 1, the tablets showed the following release profile: release lower than 20% after 2 hours; at the dissolution test at pH ≥ 6.4 they showed the following release profile: not more than 40% after 1 hour, at pH 7.2 not more than 60% after 1 hour, and not more than 70% after 2 hours; the value must be > 80% after 8 hours; and 100% after 12 hours.

### Example 19

The process is conducted similarly to Example 13. The coating is performed with shellac together with HPMC E5 Premium.

At the dissolution test at pH 1, the tablets showed the following release profile: release lower than 1% after 2 hours; at the dissolution test at pH ≥ 6.4 they showed the following release profile: not more than 20% after 1 hour, at pH 7.2 not more than 60% after 1 hour, and not more than 70% after 2 hours; the value must be > 80% after 8 hours; and 100% after 12 hours.

### Example 20

7.5 Kg of rifamycin sv sodium salt, 250 g of hydroxypropyl methylcellulose (HPMC K4M), 125 g of hydroxypropyl methylcellulose (HPMC K100M), 1.11 Kg of microcrystalline cellulose, 90 g of talc, 75 g of Mg stearate, 60 g of silicon dioxide, 10 g of polymethacrylate RL100, 10 g of polymethacrylate RS 100, 285 g of crospovidone and 285 g of croscarmellose are standardised through a 12 mesh screen.

After normalisation, 3.75 Kg of rifamycin sv sodium salt, 250 g of hydroxypropyl methylcellulose (HPMC K4M), 125 g of hydroxypropyl methylcellulose (HPMC K100M), 775 g of microcrystalline cellulose, 7.5 g of talc, 10 g of Mg stearate, 5 g of silicon dioxide, 10 g of polymethacrylate RL100, 10 g of polymethacrylate RS 100 and 200 g of ascorbic acid are taken up, placed in a mixer and mixed for 15 minutes; then loaded into a compacter; suitably compacted and standardised through a 12 mesh screen.

7.5 g of talc, 10 g of Mg stearate and 5 g of silicon dioxide are added to this mixture. The mixture is then homogenised for at least 15 minutes. This mixture will form part of the first, controlled-release layer of the mini-tablet, consisting of a single 55 mg layer.

3.75 Kg of rifaximin, 335 g of microcrystalline cellulose, 285 g of crospovidone, 285 g of croscarmellose, 37.5 g of talc, 10 g of Mg stearate and 25 g of silicon dioxide are taken up separately, placed in a mixer and mixed for 15 minutes; then loaded into a compacter; suitably compacted and standardised through a 12 mesh screen.

37.5 g of talc, 10 g of Mg stearate and 25 g of silicon dioxide are added to this mixture. The mixture is then homogenised for at least 15 minutes. This mixture will form part of the second, immediate-release layer of the mini-tablet, which comprises a single 50 mg layer.

The two separate mixtures are then compressed to obtain a 5 mm double-layer mini-tablet weighing 109 mg. The resulting mini-tablets are then film-coated with a solution/suspension containing 400 g of HPMC E5 Premium, 200 g of talc, 60 g of titanium dioxide and 20 g of triethyl citrate, to obtain a mini-tablet with a mean weight of 109 mg.

The mini-tablets, at dissolution tests at pH 1 after 1 hour, exhibit release below 50%; at pH ≥ 6.4 they showed release ≤ 60% after 1 hour; at pH 7.2 release ≤ 70% after 1 hour; release ≤ 80% after 6 hours, not more than 85% after 8 hours; the value must be 100% after 12 hours.

### Example 21

25 Kg of metronidazole, 2.1 Kg of hydroxypropyl methylcellulose (HPMC 1001v), 1.1 Kg of hydroxypropyl methylcellulose (HPMC K100M), 1.65 Kg of microcrystalline cellulose, 165 g of talc, 230 g of glyceryl palmitostearate, 165 g of silicon dioxide, 90 g of polymethacrylate L100 and 90 g of polymethacrylate S 100 are standardised through a 12 mesh screen. After normalisation, 25 Kg of metronidazole, 1.05 Kg of hydroxypropyl methylcellulose (HPMC 1001v), 55 g of hydroxypropyl methylcellulose (HPMC K100M), 825 g of microcrystalline cellulose, 82.5 g of talc, 115 g of glyceryl palmitostearate, 82.5 g of silicon dioxide, 45 g of polymethacrylate L100 and 45 g of polymethacrylate S 100 are taken up, placed in a mixer and mixed for 15 minutes; then loaded into a compacter; suitably compacted and standardised through a 12 mesh screen. An external phase comprising 1.05 Kg of hydroxypropyl methylcellulose (HPMC 1001v), 55 g of hydroxypropyl methylcellulose (HPMC K100M), 825 g of microcrystalline cellulose, 82.5 g of talc, 11.5 g of glyceryl palmitostearate, 82.5 g of silicon dioxide, 45 g of polymethacrylate L100 and 45 g of polymethacrylate S 100 is added to said mixture; and the two phases are mixed. The mixture is then compressed in a tablet press to obtain tablets weighing 305.9 mg each.

The resulting tablets are film-coated with a gastroresistant solution/suspension based on 810 g of polymethacrylate L100, 810 g of polymethacrylate S100, 50 g of talc, 60 g of titanium dioxide and 23 g of triethyl citrate, to obtain a tablet with a mean weight of 330 mg.

At disintegration and dissolution tests at pH 1, the tablets remain intact for at least 2 hours, with release below 1%; at the dissolution test: at pH ≥ 6.4 the following releases were observed: not more than 10% after 1 hour, at pH 7.2 not more than 40% after 1 hour, and not more than 50% after 2 hours; not more than 70% after 6 hours; and 100% after 12 hours.

### Example 22

The process is conducted similarly to Example 21, replacing HPMC lv 100 with HPMC K4M; then adding polymethacrylate L100/55 in the matrix to replace polymethacrylates L100 and S100, until a 305.9 mg core is obtained. The coating is performed by replacing polymethacrylates L100 and S100 with polymethacrylate L100/55 above the ethylcellulose polymer until a 365 mg tablet is obtained. At disintegration and dissolution tests at pH 1, the tablets remain intact for at least 2 hours, with release below 1%; at the dissolution test: at pH ≥ 6.4 the following releases were observed: not more than 5% after 1 hour, at pH 7.2 not more than 10% after 1 hour, and not more than 20% after 2 hours; not more than 50% after 6 hours; and 100% after 12 hours.

### Example 23

50 Kg of ciprofloxacin, 4.35 kg of hydroxypropyl methylcellulose (HPMC 100 Iv), 2.18 Kg of hydroxypropyl methylcellulose (HPMC K100M), 1.65 kg of microcrystalline cellulose, 140 g of talc, 230 g of glyceryl palmitostearate, 150 g of silicon dioxide, 90 g of polymethacrylate RL100 and 90 g of polymethacrylate RS 100 are standardised through a 12 mesh screen. After normalisation, 50 Kg of ciprofloxacin, 2.175 Kg of hydroxypropyl methylcellulose (HPMC 1001v), 1.09 Kg of hydroxypropyl methylcellulose (HPMC K100M), 825 g of microcrystalline cellulose, 70 g of talc, 115 g of glyceryl palmitostearate, 75 g of silicon dioxide, 45 g of polymethacrylate RL100 and 45 g of polymethacrylate RS 100 are taken up, placed in a mixer and mixed for 15 minutes; then loaded into a compacter; suitably compacted and standardised through a 12 mesh screen. An external phase comprising 2.175 Kg of hydroxypropyl methylcellulose (HPMC 1001v), 1.09 Kg of hydroxypropyl methylcellulose (HPMC K100M), 825 g of microcrystalline cellulose, 70 g of talc, 115 g of glyceryl palmitostearate, 75 g of silicon dioxide, 45 g of polymethacrylate RL100 and 45 g of polymethacrylate RS 100 is added to said mixture; and the two phases are mixed. The mixture is then compressed in a tablet press to obtain tablets weighing 589 mg each.

The resulting tablets are then film-coated with a gastroresistant solution/suspension based on 6 Kg of polymethacrylate L100/55, 1.58 Kg of HPMC E5P, 50 g of talc, 40 g of titanium dioxide and 52 g of triethyl citrate, to obtain a tablet with a mean weight of 679 mg.

At disintegration and dissolution tests at pH 1, the tablets remain intact for at least 2 hours, with release below 1%; at the dissolution test: at pH ≥ 6.4 the following releases were observed: not more than 10% after 1 hour, at pH 7.2 not more than 15% after 1 hour, and not more than 25% after 2 hours; not more than 65% after 6 hours; and 100% after 12 hours.

### Example 24

The process is conducted similarly to Example 23. The coating is performed by replacing polymethacrylate L1005 with HPMC E5P to obtain a 678 mg tablet. At disintegration and dissolution tests at pH 1, the tablets remain intact for at least 2 hours, with release below 10%; at the dissolution test: at pH ≥ 6.4 the following releases were observed: not more than 15% after 1 hour, at pH 7.2 not more than 20% after 1 hour, and not more than 30% after 2 hours; not more than 50% after 6 hours; and 100% after 12 hours.

### Example 25

25.6 Kg of vancomycin HCl (equal to 25 kg of vancomycin base), 2.5 kg of hydroxypropyl methylcellulose (HPMC K4M), 2.5 Kg of hydroxypropyl methylcellulose (HPMC K100M), 10 kg of microcrystalline cellulose, 200 g of talc, 500 g of glyceryl palmitostearate, 300 g of silicon dioxide, 100 g of polymethacrylate RL100 and 100 g of polymethacrylate RS 100 are standardised through a 12 mesh screen. After normalisation, 25.6 Kg of vancomycin HCl, 12.5 Kg of hydroxypropyl methylcellulose (HPMC K4M), 12.5 Kg of hydroxypropyl methylcellulose (HPMC K100M), 5 kg of microcrystalline cellulose, 100 g of talc, 250 g of glyceryl palmitostearate, 150 g of silicon dioxide, 50 g of polymethacrylate RL100 and 50 g of polymethacrylate RS 100 are taken up, placed in a mixer and mixed for 15 minutes; then loaded into a compacter; suitably compacted and standardised through a 12 mesh screen.

An external phase comprising 12.5 Kg of hydroxypropyl methylcellulose (HPMC K4M), 12.5 Kg of hydroxypropyl methylcellulose (HPMC K100M), 5 Kg of microcrystalline cellulose, 100 g of talc, 250 g of glyceryl palmitostearate, 150 g of silicon dioxide, 50 g of polymethacrylate RL100 and 50 g of polymethacrylate RS 100 is added to said mixture; and the two phases are mixed. The mixture is then compressed in a tablet press to obtain tablets weighing 418 mg each.

The resulting tablets are then film-coated with a suspension based on 2.4 Kg of HPMC E5P, 10 g of talc, 10 g of titanium dioxide and 40 g of triethyl citrate, to obtain a tablet with a mean weight of 448 mg.

At disintegration and dissolution tests at pH 1, the tablets remain intact for at least 2 hours, with release below 1%; at the dissolution test: at pH ≥ 6.4 the following releases were observed: not more than 5% after 1 hour, at pH 7.2 not more than 10% after 1 hour, and not more than 15% after 2 hours; not more than 60% after 6 hours; and 100% after 12 hours.

### Example 26

8.445 Kg of amoxicillin, 1.104 kg of rifabutin, 0.883 Kg of omeprazole, 0.4 kg of hydroxypropyl methylcellulose (HPMC K4M), 0.2 Kg of hydroxypropyl methylcellulose (HPMC K15M), 8.468 Kg of microcrystalline cellulose, 1 Kg of talc, 1.4 Kg of glyceryl palmitostearate, 1 Kg of silicon dioxide, 100 g of polymethacrylate L100/55, 1 Kg of crospovidone and 1 Kg of croscarmellose are standardised through a 12 mesh screen.

After normalisation, 8.445 Kg of amoxicillin, 1.104 kg of rifabutin, 0.4 kg of hydroxypropyl methylcellulose (HPMC K4M), 0.2 Kg of hydroxypropyl methylcellulose (HPMC K15M), 4.718 Kg of microcrystalline cellulose, 0.5 kg of talc, 0.7 Kg of glyceryl palmitostearate, 0.5 Kg of silicon dioxide and 100 g of polymethacrylate L100/55 are taken up, placed in a mixer and mixed for 15 minutes; then loaded into a compacter; suitably compacted and standardised through a 12 mesh screen.

0.5 kg of talc, 0.7 Kg of glyceryl palmitostearate and 0.5 Kg of silicon dioxide are added to this mixture. The mixture is then homogenised for at least 15 minutes. This mixture will form part of the first, controlled-release layer of the mini-tablet, consisting of a single 16.667 mg layer.

0.883 Kg of omeprazole, 3.75 Kg of microcrystalline cellulose, 1 Kg of crospovidone, 1 Kg of croscarmellose, 0.25 kg of talc, 0.35 Kg of glyceryl palmitostearate and 0.25 Kg of silicon dioxide are taken up separately, placed in a mixer and mixed for 15 minutes; then loaded into a compacter; suitably compacted and standardised through a 12 mesh screen.

0.25 kg of talc, 0.35 Kg of glyceryl palmitostearate and 0.25 Kg of silicon dioxide are added to this mixture. The mixture is then homogenised for at least 15 minutes. This mixture will form part of the second, immediate-release layer of the mini-tablet, which comprises a single 8.33 mg layer.

The two separate mixtures are then compressed to obtain a 3 mm double-layer mini-tablet weighing 25 mg. The resulting mini-tablets are then film-coated with a solution/suspension containing 9 Kg of HPMC L100/55, 100 g of talc, 200 g of titanium dioxide and 500 g of triethyl citrate, to obtain a mini-tablet with a mean weight of 35 mg.

At dissolution tests at pH 1 for amoxicillin, the mini-tablets exhibit release below 1% after 2 hours; at pH ≥ 5.5 they showed release ≤ 10% after 1 hour; at pH 7.2 release ≤ 30% after 1 hour; release ≤ 60% after 6 hours, release ≤85% after 8 hours; the value must be 100% after 12 hours.

At dissolution tests at pH 1 for rifabutin, the mini-tablets exhibit release below 1% after 2 hours; at pH ≥ 5.5 they showed release ≤ 10% after 1 hour; at pH 7.2 release ≤ 25% after 1 hour; release ≤ 50% after 6 hours, release ≤70% after 8 hours; the value must be 100% after 12 hours.

At dissolution tests at pH 1 for omeprazole, the mini-tablets exhibit release below 1% after 2 hours; at pH ≥ 5.5 they showed release ≤ 10% after 1 hour; at pH 7.2 release ≤ 80% after 1 hour; the value must be 100% after 6 hours.

### Example 27

The process is conducted similarly to Example 26, replacing hydroxypropyl methylcellulose (HPMC K4M) with hydroxypropyl methylcellulose (HPMC 1001v), and hydroxypropyl methylcellulose (HPMC K100M) with hydroxypropyl methylcellulose (HPMC K15M), in the core containing amoxicillin and rifabutin.

At dissolution tests at pH 1 for amoxicillin, the mini-tablets exhibit release below 1% after 2 hours; at pH ≥ 5.5 they showed release ≤ 10% after 1 hour; at pH 7.2 release ≤ 25% after 1 hour; release ≤ 55% after 6 hours, release ≤70% after 8 hours; the value must be 100% after 12 hours.

At dissolution tests at pH 1 for rifabutin, the mini-tablets exhibit release below 1% after 2 hours; at pH ≥ 5.5 they showed release ≤ 10% after 1 hour; at pH 7.2 release ≤ 20% after 1 hour; release ≤ 65% after 6 hours, release ≤80% after 8 hours; the value must be 100% after 12 hours.

At dissolution tests at pH 1 for omeprazole, the mini-tablets exhibit release below 5% after 2 hours; at pH ≥ 5.5 they showed release ≤ 10% after 1 hour; at pH 7.2 release ≤ 80% after 1 hour; the value must be 100% after 6 hours.

### Example 28

The process is conducted similarly to Example 27, replacing hydroxypropyl methylcellulose (HPMC 1001v) with hydroxypropyl methylcellulose (HPMC K4M), and hydroxypropyl methylcellulose (HPMC K100) with hydroxypropyl methylcellulose (HPMC K15M), in the core containing amoxicillin and rifabutin. Shellac is also added, both in the core and in the film-coating, instead of polymethacrylate L100/55.

At dissolution tests at pH 1 for amoxicillin, the mini-tablets exhibit release below 1% after 2 hours; at pH ≥ 5.5 they showed release ≤ 10% after 1 hour; at pH 7.2 release ≤ 30% after 1 hour; release ≤ 60% after 6 hours, release ≤80% after 8 hours; the value must be 100% after 12 hours.

At dissolution tests at pH 1 for rifabutin, the mini-tablets exhibit release below 1% after 2 hours; at pH ≥ 5.5 they showed release ≤ 10% after 1 hour; at pH 7.2 release ≤ 30% after 1 hour; release ≤ 70% after 6 hours, release ≤90% after 8 hours; the value must be 100% after 12 hours.

At dissolution tests at pH 1 for omeprazole, the mini-tablets exhibit release below 5% after 2 hours; at pH ≥5.5 they showed release ≤ 10% after 1 hour; at pH 7.2 release ≤ 80% after 1 hour; the value must be 100% after 6 hours.

### Example 29

The process is conducted similarly to Example 28, replacing hydroxypropyl methylcellulose (HPMC K4M) with hydroxypropyl methylcellulose (HPMC 1001v), and hydroxypropyl methylcellulose (HPMC K15M) with hydroxypropyl methylcellulose (HPMC K100M), in the core containing amoxicillin and rifabutin. Polymethacrylate L100/55 is also added.

At dissolution tests at pH 1 for amoxicillin, the mini-tablets exhibit release below 1% after 2 hours; at pH ≥ 5.5 they showed release ≤ 10% after 1 hour; at pH 7.2 release ≤ 40% after 1 hour; release ≤ 70% after 6 hours, release ≤85% after 8 hours; the value must be 100% after 12 hours.

At dissolution tests at pH 1 for rifabutin, the mini-tablets exhibit release below 1% after 2 hours; at pH ≥ 5.5 they showed release ≤ 10% after 1 hour; at pH 7.2 release ≤ 35% after 1 hour; release ≤ 80% after 6 hours, release ≤85% after 8 hours; the value must be 100% after 12 hours.

At dissolution tests at pH 1 for omeprazole, the mini-tablets exhibit release below 5% after 2 hours; at pH ≥ 5.5 they showed release ≤ 10% after 1 hour; at pH 7.2 release ≤ 80% after 1 hour; the value must be 100% after 6 hours.

### Comparative Example 30

Comparison tests of the dissolution profiles were performed, reproducing menthol tablets according to the formulas of Examples 2 (Ex2A) and 3 (Ex3 B) of WO 2015/087258 (characterised by the presence of two HPMCs in the core) vs. tablets formulated with the complex matrix of the invention, on each occasion inserting pH-dependent polymethacrylates L100, S100 (Ex2 A1) (Ex3 B1), shellac (Ex2 A2) (Ex3 B2), cellulose acetate phthalate (Ex2A3) (Ex3 B3), and pH-independent polymethacrylates RL100, RS100 (Ex2 A4) (Ex3 B4). The composition of the formulations in question is shown in the tables below:

Formulations according to WO 2015/087258

| | mg/tablet Ex1 F1 | mg/tablet Ex2 F2 A | mg/tablet Ex3 F3 B | mg/tablet Ex4 F4 | mg/tablet Ex5 F5 |
|---|---|---|---|---|---|
| Core | | | | | |
| Menthol | 50 | 50 | 200 | 200 | 500 |
| Dibasic calcium phosphate | 200 | 210 | - | - | 350 |
| Microcrystalline cellulose | 200 | 50 | 63 | 63 | - |
| Mannitol | 150 | 55 | 120 | 120 | - |
| HPMC K1001v | 150 | 100 | 50 | 50 | - |
| HPMC K4M | - | 55 | - | - | - |
| HPMC K15M | - | - | 50 | | 150 |
| Polyoxyethylene oxide | - | - | - | 50 | |
| Lecithin | 8 | - | 10 | 10 | - |
| Crospovidone | 2 | - | - | - | 2 |
| Croscarmellose | - | - | 2 | 2 | - |
| Mg stearate | 5 | 3 | 5 | 5 | 5 |
| Glyceryl behenate | - | - | - | - | 8 |
| Silicon dioxide | 5 | 5 | 10 | 10 | 5 |
| Total | 770 | 528 | 510 | 510 | 1020 |
| Film Coating | | | | | |
| Shellac | 30 | 20 | 43.5 | - | 160 |
| HPMC 5 P | 12 | 8 | 4.3 | - | 20 |
| Glycerin | 6 | 2 | 2.2 | - | 8 |
| Ethylcellulose | - | - | - | 20 | - |
| Sodium alginate | - | - | - | 4 | - |
| Titanium dioxide | - | - | - | 0.5 | - |
| Total | 48 | 30 | 50 | 25 | 188 |
| Grand total | 818 | 558 | 560 | 535 | 1208 |

Menthol formulations according to the invention: pH-dependent Eudragit

| | mg/tablet Ex1 F1 | mg/tablet Ex2 F2 A1 | mg/tablet Ex3 F3 B1 | mg/tablet Ex4 F4 | mg/tablet Ex5 F5 |
|---|---|---|---|---|---|
| Core | | | | | |
| Menthol | 50 | 50 | 200 | 200 | 500 |
| Dibasic calcium phosphate | 198 | 207 | - | - | 347 |
| Microcrystalline cellulose | 200 | 50 | 61.5 | 62 | - |
| Mannitol | 150 | 55 | 120 | 120 | - |
| HPMC K1001v | 150 | 100 | 50 | 50 | - |
| HPMC K4M | - | 55 | - | - | - |
| HPMC K15M | - | - | 50 | - | 150 |
| Eudragit L100 | 1.5 | 1.5 | 0.75 | 0.5 | 1.5 |
| Eudragit S100 | 1.5 | 1.5 | 0.75 | 0.5 | 1.5 |
| Polyoxyethylene oxide | - | - | - | 50 | - |
| Lecithin | 8 | - | 10 | 10 | - |
| Crospovidone | 2 | - | - | - | 2 |
| Croscarmellose | - | - | 2 | 2 | - |
| Mg stearate | 5 | 3 | 5 | 5 | 5 |
| Glyceryl behenate | - | - | - | - | 8 |
| Silicon dioxide | 5 | 5 | 10 | 10 | 5 |
| Total | 770 | 528 | 510 | 510 | 1020 |

| | | | | | |
|---|---|---|---|---|---|
| Film Coating | | | | | |
| Shellac | 30 | 20 | 43.5 | - | 160 |
| HPMC 5 P | 12 | 8 | 4.3 | - | 20 |
| Glycerin | 6 | 2 | 2.2 | - | 8 |
| Ethylcellulose | - | - | - | 20 | - |
| Sodium alginate | - | - | - | 4 | - |
| Titanium dioxide | - | - | - | 0.5 | - |
| Total | 48 | 30 | 50 | 25 | 188 |
| Grand total | 818 | 558 | 560 | 535 | 1208 |

Menthol formulations according to the invention: shellac

| | mg/tablet Ex1 F1 | mg/tablet Ex2 F2 A2 | mg/tablet Ex3 F3 B2 | mg/tablet Ex4 F4 | mg/tablet Ex5 F5 |
|---|---|---|---|---|---|
| Core | | | | | |
| Menthol | 50 | 50 | 200 | 200 | 500 |
| Dibasic calcium phosphate | 198 | 207 | - | - | 347 |
| Microcrystalline cellulose | 200 | 50 | 61.5 | 62 | - |
| Mannitol | 150 | 55 | 120 | 120 | - |
| HPMC K1001v | 150 | 100 | 50 | 50 | - |
| HPMC K4M | - | 55 | - | - | - |
| HPMC K15M | - | - | 50 | - | 150 |
| Shellac | 3 | 3 | 1.5 | 1 | 3 |
| Polyoxyethylene oxide | - | - | - | 50 | - |
| Lecithin | 8 | - | 10 | 10 | - |
| Crospovidone | 2 | - | - | - | 2 |
| Croscarmellose | - | - | 2 | 2 | - |
| Mg stearate | 5 | 3 | 5 | 5 | 5 |
| Glyceryl behenate | - | - | - | - | 8 |
| Silicon dioxide | 5 | 5 | 10 | 10 | 5 |
| Total | 770 | 528 | 510 | 510 | 1020 |
| Film Coating | | | | | |
| Shellac | 30 | 20 | 43.5 | - | 160 |
| HPMC 5 P | 12 | 8 | 4.3 | - | 20 |
| Glycerin | 6 | 2 | 2.2 | - | 8 |
| Ethylcellulose | - | - | - | 20 | - |
| Sodium alginate | - | - | - | 4 | - |
| Titanium dioxide | - | - | - | 0.5 | - |
| Total | 48 | 30 | 50 | 25 | 188 |
| Grand total | 818 | 558 | 560 | 535 | 1208 |

Menthol formulations according to the invention: pH-dependent cellulose acetate phthalate

| | mg/tablet Ex1 F1 | mg/tablet Ex2 F2 A3 | mg/tablet Ex3 F3 B3 | mg/tablet Ex4 F4 | mg/tablet Ex5 F5 |
|---|---|---|---|---|---|
| Core | | | | | |
| Menthol | 50 | 50 | 200 | 200 | 500 |
| Dibasic calcium phosphate | 198 | 207 | - | - | 347 |
| Microcrystalline cellulose | 200 | 50 | 61.5 | 62 | - |
| Mannitol | 150 | 55 | 120 | 120 | - |
| HPMC K1001v | 150 | 100 | 50 | 50 | - |
| HPMC K4M | - | 55 | - | - | - |
| HPMC K15M | - | - | 50 | - | 150 |
| Cellulose acetate phthalate | 3 | 3 | 1.5 | 1 | 3 |
| Polyoxyethylene oxide | - | - | - | 50 | - |
| Lecithin | 8 | - | 10 | 10 | - |
| Crospovidone | 2 | - | - | - | 2 |
| Croscarmellose | - | - | 2 | 2 | |
| Mg stearate | 5 | 3 | 5 | 5 | 5 |
| Glyceryl behenate | - | - | - | - | 8 |
| Silicon dioxide | 5 | 5 | 10 | 10 | 5 |
| Total | 770 | 528 | 510 | 510 | 1020 |
| Film Coating | | | | | |
| Shellac | 30 | 20 | 43.5 | - | 160 |
| HPMC 5 P | 12 | 8 | 4.3 | - | 20 |
| Glycerin | 6 | 2 | 2.2 | - | 8 |
| Ethylcellulose | - | - | - | 20 | - |
| Sodium alginate | - | - | - | 4 | - |
| Titanium dioxide | - | - | - | 0.5 | - |
| Total | 48 | 30 | 50 | 25 | 188 |
| Grand total | 818 | 558 | 560 | 535 | 1208 |

Menthol formulations according to the invention: pH-independent Eudragit

| Raw material | mg/tablet Ex1 F1 | mg/tablet Ex2 F2 A4 | mg/tablet Ex3 F3 B4 | mg/tablet Ex4 F4 | mg/tablet Ex5 F5 |
|---|---|---|---|---|---|
| Core | | | | | |
| Menthol | 50 | 50 | 200 | 200 | 500 |
| Dibasic calcium phosphate | 198 | 207 | - | - | 347 |
| Microcrystalline cellulose | 200 | 50 | 61.5 | 62 | - |
| Mannitol | 150 | 55 | 120 | 120 | - |
| HPMC K1001v | 150 | 100 | 50 | 50 | - |
| HPMC K4M | - | 55 | - | - | - |
| HPMC K15M | - | - | 50 | - | 150 |
| Eudragit LS100 | 1.5 | 1.5 | 0.75 | 0.5 | 1.5 |
| Eudragit RS 100 | 1.5 | 1.5 | 0.75 | 0.5 | 1.5 |
| Polyoxyethylene oxide | - | - | - | 50 | - |
| Lecithin | 8 | - | 10 | 10 | - |
| Crospovidone | 2 | - | - | - | 2 |
| Croscarmellose | - | - | 2 | 2 | - |
| Mg stearate | 5 | 3 | 5 | 5 | 5 |
| Glyceryl behenate | - | - | - | - | 8 |
| Silicon dioxide | 5 | 5 | 10 | 10 | 5 |
| Total | 770 | 528 | 510 | 510 | 1020 |
| Film Coating | | | | | |
| Shellac | 30 | 20 | 43.5 | - | 160 |
| HPMC 5 P | 12 | 8 | 4.3 | - | 20 |
| Glycerin | 6 | 2 | 2.2 | - | 8 |
| Ethylcellulose | - | - | - | 20 | - |
| Sodium alginate | - | - | - | 4 | - |
| Titanium dioxide | - | - | - | 0.5 | - |
| Total | 48 | 30 | 50 | 25 | 188 |
| Grand total | 818 | 558 | 560 | 535 | 1208 |

The annexed Figures show significant differences between the formulations of the invention and those described in WO 2015/087258 as regards the homogeneity of the data (very low %RSD for the formulas of the invention), and above all, very similar profiles even at different pH values.

### BRIEF DESCRIPTION OF FIGURES

FIG. 1 a-d: dissolution profiles of the formulations according to WO 2015/087258.

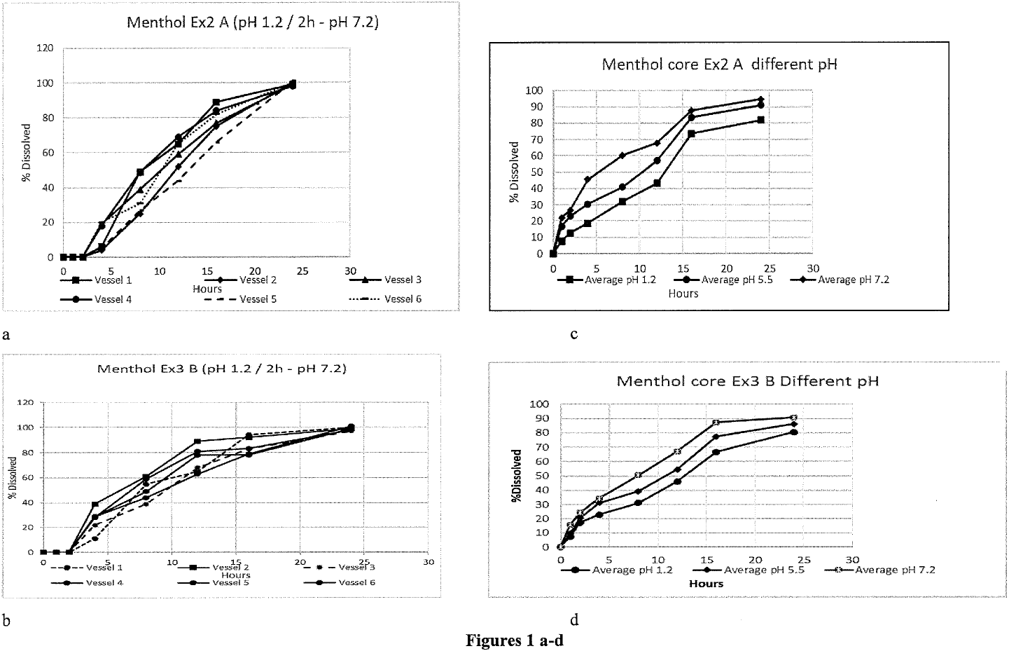

FIG. 2 a-d: dissolution profiles of the formulations comprising pH-dependent Eudragit in the matrix containing HPMC.

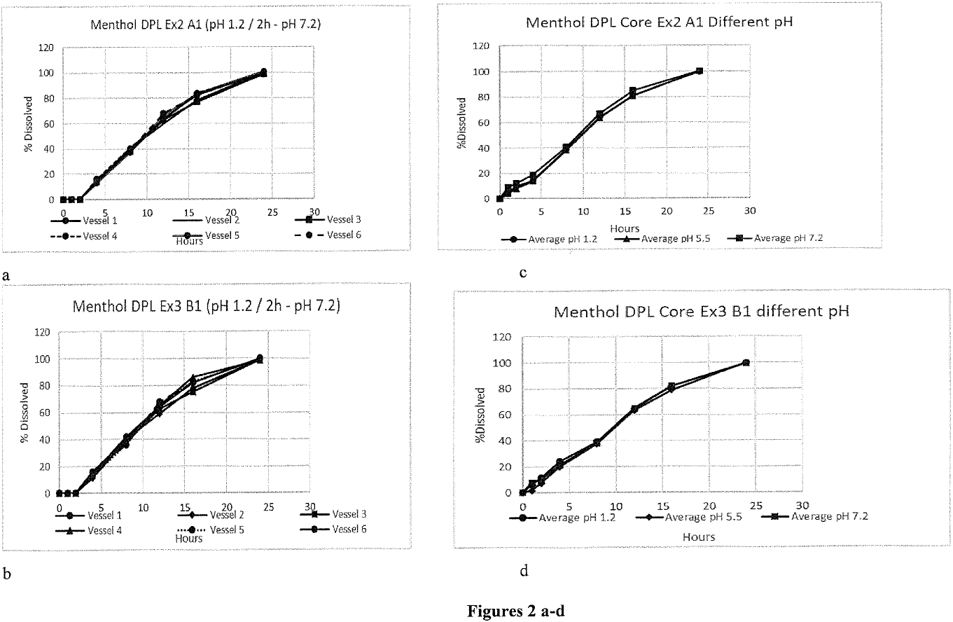

FIG. 3 a-d: dissolution profiles of the formulations comprising shellac in the matrix containing HPMC.

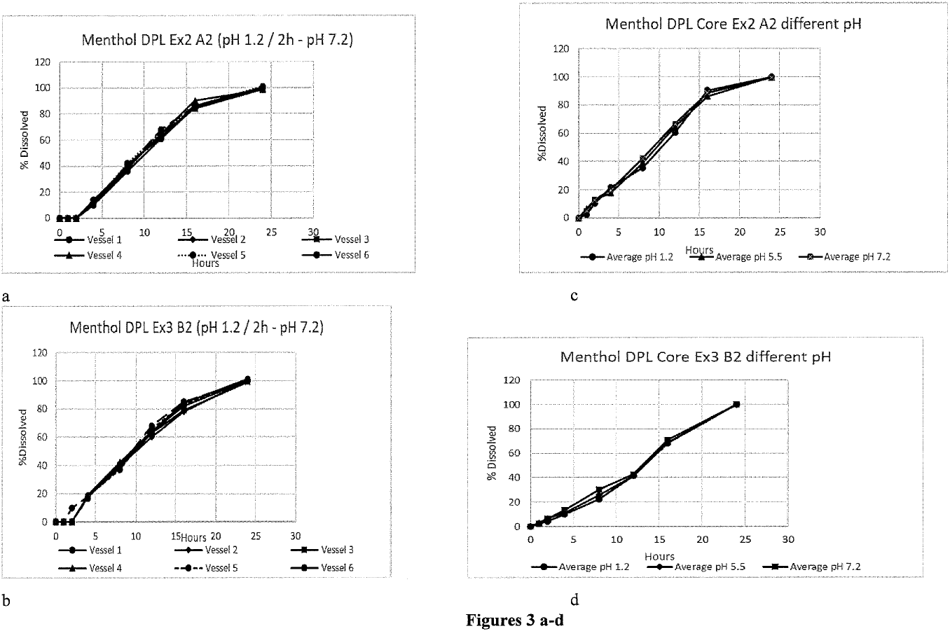

FIG. 4 a-d: dissolution profiles of the formulations comprising cellulose acetate phthalate in the matrix containing HPMC.

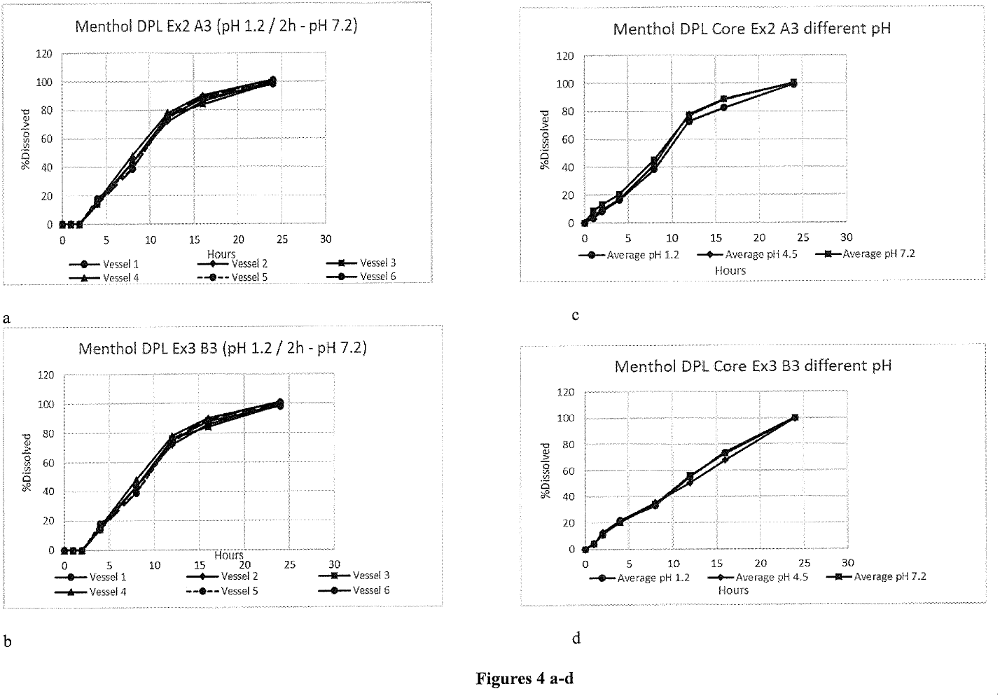

FIG. 5 a-d: dissolution profiles of the formulations comprising pH-independent Eudragit in the matrix containing HPMC.

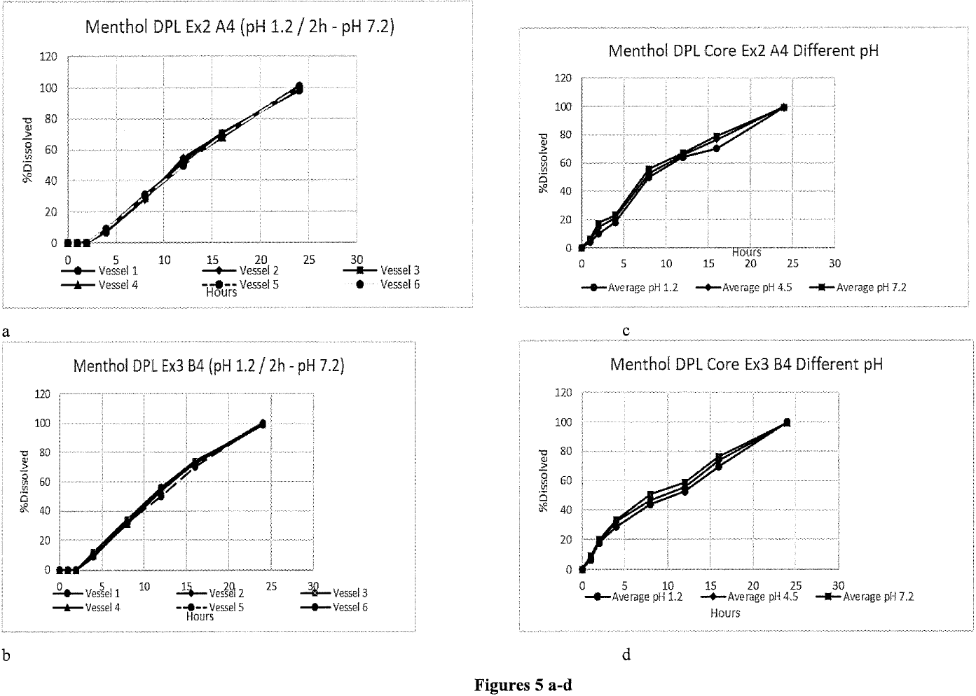

### RESULTS

The results demonstrate that the addition of polymethacrylates, shellac or cellulose acetate phthalate to the matrix containing the two HPMCs with different viscosities gives rise to very similar dissolution profiles (%RSD ≤ 3% for each control point) and independence from the pH of the dissolution bath (pH 1.2, 5.5, 7.2), which are not possessed by the menthol formulations of the prior art without the complex matrix. Very similar results were obtained with the formulations of Examples 1-29.

1. A controlled-release solid oral pharmaceutical composition comprising
an antibacterial/antibiotic for the treatment of intestinal infections in a core and an outer coating of said core, wherein :
a) the core comprises:
(i) a monolithic matrix containing the antibacterial/antibiotic, at least one hydroxypropyl methylcellulose having a viscosity ranging between 3 and 5000 mPa.s 2% in H2O at 20° C., at least one hydroxypropyl methylcellulose having a viscosity ranging between 13500 and 280000 mPa.s 2% in H2O at 20° C., at least one or two methacrylic polymers/copolymers and/or shellac, cellulose acetate phthalate, hydroxypropyl methylcellulose acetate succinate, or
(ii) a monolithic matrix as defined above adjacent to an immediate-release layer comprising the same antibacterial/antibiotic as contained in the monolithic matrix;
b) the outer coating comprises a layer comprising ethylcellulose or a gastroresistant layer or a layer comprising ethylcellulose which in turn is coated with gastroresistant polymers.

2. The composition as claimed in claim 1, wherein the core comprises a monolithic matrix as defined in claim 1, point (i).

3. The composition as claimed in claim 1, wherein the core comprises a monolithic matrix as defined in claim 1, wherein said monolithic matrix is adjacent to an immediate-release layer, and wherein said monolithic matrix and said immediate-release layer comprise the antibacterial agent .

4. The composition as claimed in claim 1, wherein the coating comprises a layer comprising ethylcellulose.

5. The composition as claimed in claim 1, wherein the coating comprises a layer comprising ethylcellulose coated with gastroresistant polymers.

6. The composition as claimed in claim 1, wherein the coating comporises a gastroresistant layer.

7. The composition as claimed in claim 1, wherein the methacrylic acid polymer or copolymer is selected from the group consisting of pH-independent methacrylic ester copolymers, pH-independent ammonium alkyl methacrylate copolymers; amino alkyl methacrylate copolymers soluble at pH equal or less than 5.0, methacrylic acid copolymers soluble at pH equal or above 5.5, methacrylic acid copolymers soluble at pH 6.0-7.0 and pH-dependent methacrylic acid copolymers soluble at pH equal or above 7.0.

8. The composition as claimed in claim 1, wherein the monolithic matrix comprises shellac.

9. The composition as claimed in claim 1, wherein the gastroresistant coating comprises pH-dependent methacrylic acid copolymers soluble at pH equal or above 5.5; pH-dependent methacrylic acid copolymers soluble at pH 6.0-7.0; pH-dependent methacrylic acid copolymers soluble at pH equal or above 7.0; shellac; cellulose acetate phthalate; cellulose succinate.

10. The composition as claimed in claim 1, wherein the hydroxypropyl methylcellulose having a viscosity ranging between 3 and 5000 mPa.s 2% in H2O at 20° C. constitutes 1 to 20% of the weight of the core, the hydroxypropyl methylcellulose having a viscosity ranging between 13500 and 280,000 mPa.s 2% in H2O at 20° C. constitutes 1 to 20% of the weight of the matrix, and the methacrylic polymer/copolymer constitutes 0.1 to 2% of the weight of the core.

11. The composition as claimed in claim 1, wherein ethylcellulose is present in an amount of 1 to 20% of the weight of the core.

12. The composition as claimed in claim 1, wherein the antibacterial/antibiotic for the treatment of intestinal infections is selected from the group consisting of rifaximin, rifampicin, rifamycin SV and salts thereof, oral beta lactam antibiotics, macrolides, quinolones and metronidazole.
